SEQUENCE LISTING (3 sequences)

SEQ ID NO: 1 - protein, 40 aa, Homo sapiens
DAEFRHDSGYEVHHQKLVFFAEDVGSNKGAIIGLMVGGVV

SEQ ID NO: 2 - protein, 42 aa, Homo sapiens [sequence omitted]

SEQ ID NO: 3 - protein, 8 aa, synthetic construct
DAEFRHDS

(19) United States
(12) Patent Application Publication
(10) Pub. No.: US 2026/0298944 A1
(43) Pub. Date: Oct. 1, 2026

(54) METHOD FOR MEASURING POLYPEPTIDE AGGREGATE, CALIBRATOR, COMPLEX, AND METHOD FOR PRODUCING THE SAME

(71) Applicant: SYSMEX CORPORATION, Hyogo (JP)

(72) Inventors: Ishita Gupta, Hyogo (JP); Hui Zhou, Hyogo (JP); Kazuya Matsumoto, Hyogo (JP); Masahiro Miura, Hyogo (JP)

(73) Assignee: SYSMEX CORPORATION, Hyogo (JP)

(21) Appl. No.: 19/572,877

(22) Filed: Mar. 20, 2026

(30) Foreign Application Priority Data
Mar. 28, 2025 (JP) 2025-056315

(51) Int. Cl. G01N 33/68; G01N 33/543

(52) U.S. Cl. CPC G01N 33/6878; G01N 33/54346; G01N 2333/4709

(57) ABSTRACT

Provided is a method for measuring a polypeptide aggregate in a sample, wherein the method comprises obtaining information regarding a concentration of the polypeptide aggregate in the sample using a calibrator, and the calibrator comprises a complex comprising: an oligopeptide comprising an epitope of the polypeptide aggregate; a hydrophilic polymer chain not comprising the epitope; and a carrier particle.

### CROSS REFERENCE TO RELATED APPLICATIONS

This application claims priority from prior Japanese Patent Application No. 2025-056315, filed on Mar. 28, 2025, entitled “METHOD FOR MEASURING A POLYPEPTIDE AGGREGATE, CALIBRATOR, COMPLEX, AND METHOD FOR PRODUCING THE SAME”, the entire content of which is incorporated herein by reference.

### REFERENCE TO A SEQUENCE LISTING

The instant application contains a Sequencing Listing which has been submitted electronically in XML file and is hereby incorporated by reference in its entirety. Said XML copy, created on Mar. 21, 2025, is named 164499-US-seqlist and is 3,509 bytes in size.

### FIELD OF THE INVENTION

The present invention relates to a method for measuring a polypeptide aggregate in a sample. The present invention relates to a calibrator for obtaining information regarding a concentration of the polypeptide aggregate in the sample. The present invention relates to a complex for use in the method for measuring the polypeptide aggregate in the sample. The present invention relates to a method for producing a complex used for obtaining information regarding a concentration of the polypeptide aggregate in the sample.

### BACKGROUND OF THE INVENTION

Aggregation of polypeptides in vivo can cause various diseases. For example, Alzheimer's disease is considered to be caused by the accumulation of amyloid β (Aβ) aggregates in the brain. Also, Parkinson's disease is considered to be caused by the accumulation of α-synuclein aggregates in the central nervous system. Therefore, the polypeptide aggregate in a sample obtained from a living body is sometimes measured to evaluate the state of a disease caused by a polypeptide aggregate or the risk of developing the disease.

When a test substance in a sample is quantitatively measured, a calibrator is usually used. A calibrator generally refers to one or a plurality of reagents comprising a standard substance corresponding to the test substance. For example, in immunoassay measurement, the test substance itself, isolated from a living body or synthesized, is used as a standard substance. When the test substance is a polypeptide aggregate, an oligomer prepared from a monomer polypeptide constituting the aggregate is sometimes used as a standard substance. This oligomer is a molecule mimicking the polypeptide aggregate. For example, Rahimi F. et al., Photo-Induced Cross-Linking of Unmodified Proteins (PICUP) Applied to Amyloidogenic Peptides, J. Vis. Exp., vol. 23, e1071, 2009 describes that an Aβ peptide was cross-linked by Photo-Induced Cross-Linking of Unmodified Proteins (PICUP) to prepare an Aβ oligomer.

### SUMMARY OF THE INVENTION

An object of the present invention is to provide a novel standard substance suitable for the measurement of a polypeptide aggregate in a sample, a calibrator comprising the standard substance, a method for producing the standard substance, and a method for measuring using the calibrator.

The present invention provides a method for measuring a polypeptide aggregate in a sample, which comprises obtaining information regarding a concentration of the polypeptide aggregate in the sample using a calibrator, and the calibrator comprises a complex comprising: an oligopeptide comprising an epitope of the polypeptide aggregate; a hydrophilic polymer chain not comprising the epitope; and a carrier particle.

The present invention provides a calibrator for obtaining information regarding a concentration of a polypeptide aggregate in a sample, comprising a complex comprising: an oligopeptide comprising an epitope of the polypeptide aggregate; a hydrophilic polymer chain not comprising the epitope; and a carrier particle.

The present invention provides a complex for use in the above-mentioned method for measuring, comprising: an oligopeptide comprising an epitope of the polypeptide aggregate; a hydrophilic polymer chain not comprising the epitope; and a carrier particle.

The present invention provides a method for producing a complex used for obtaining information regarding a concentration of a polypeptide aggregate in a sample, comprising immobilizing an oligopeptide comprising an epitope of the polypeptide aggregate and a hydrophilic polymer chain not comprising the epitope on a surface of a carrier particle.

According to the present invention, a method for measuring a polypeptide aggregate in a sample, a calibrator for obtaining information regarding a concentration of the polypeptide aggregate, a complex for use in the method for measuring, and a method for producing the complex are provided.

### BRIEF DESCRIPTION OF THE DRAWINGS

FIG. 1 is a schematic view of the complex of the present embodiment.

FIG. 2A is a schematic view of the step of forming a first immune complex comprising the complex of the present embodiment.

FIG. 2B is a schematic view of the step of detecting the complex of the present embodiment.

FIG. 3A is a schematic view of the step of forming a second immune complex comprising the polypeptide aggregate in the sample.

FIG. 3B is a schematic view of the step of detecting the polypeptide aggregate in the sample.

FIG. 4 is a schematic view of a calibrator consisting of one reagent.

FIG. 5A is a schematic view of a calibrator which is a reagent set comprising a plurality of reagents.

FIG. 5B is a schematic view of a calibrator which is a reagent set comprising a plurality of reagents.

FIG. 6A is a calibration curve created based on the measurement results of the reagent comprising the complex of the comparative example.

FIG. 6B is a calibration curve created based on the measurement results of the calibrator (PEG chain molecular weight: 550) prepared in Example 1.

FIG. 6C is a calibration curve created based on the measurement results of the calibrator (PEG chain molecular weight: 3.4k) prepared in Example 1.

FIG. 6D is a calibration curve created based on the measurement results of the calibrator (PEG chain molecular weight: 10k) prepared in Example 1.

FIG. 6E is a calibration curve created based on the measurement results of the calibrator (PEG chain molecular weight: 20k) prepared in Example 1.

FIG. 7 is a graph showing the storage stability of the reagents comprising the various calibrators prepared in Example 1 and the complex of the comparative example.

### DETAILED DESCRIPTION

Typically, the concentration of the test substance in the sample is unknown. On the other hand, a calibrator comprises a standard substance at a predetermined concentration. Therefore, by using the calibrator, the test substance can be quantitatively measured. In the method for measuring the polypeptide aggregate in the sample of the present embodiment (hereinafter also referred to as “the method for measuring of the present embodiment”), information regarding the concentration of the polypeptide aggregate in the sample is obtained using the calibrator.

Generally, a polypeptide aggregate is a multimer or polymer composed of a plurality of monomer polypeptides of the same kind. More specifically, the polypeptide aggregate is formed by the physical or chemical polymerization of a plurality of monomer polypeptides of the same kind. Alternatively, the polypeptide aggregate is formed by the association of a plurality of monomer peptides of the same kind. In the polypeptide aggregate, the monomer polypeptides do not need to be firmly bound to each other by covalent bonds or the like. The polypeptide aggregate also includes a group of monomer polypeptides formed by weaker bonds. The polypeptide aggregate may contain molecules other than the monomer polypeptide.

The polypeptide aggregate as the test substance is not particularly limited. Preferably, it is a polypeptide aggregate existing in a living body. Examples of the polypeptide aggregate existing in a living body include amyloid. Examples of monomer polypeptides capable of forming amyloid include amyloid β (Aβ), α-synuclein, serum amyloid A protein, immunoglobulin L chain, transthyretin, β2 microglobulin, procalcitonin, prion, and tau protein.

The sample is not particularly limited as long as there is a possibility that a polypeptide aggregate is contained. A preferred sample is a biological sample. Examples of the biological sample include blood sample (whole blood), plasma, serum, cerebrospinal fluid, lymph, tissue fluid, urine, and saliva.

When the sample contains insoluble impurities such as cells, the impurities may be removed from the sample by known means such as centrifugation or filtration. The sample may be diluted with an appropriate aqueous medium, if necessary. Such an aqueous medium is not particularly limited as long as it does not interfere with the measurement described later, and examples thereof include water, physiological saline, and buffer solution. The buffer solution is preferably one having a buffering action at a nearly neutral pH (e.g., pH 6 or more and 8 or less). Examples of such a buffer solution include Good's buffers (MES, HEPES, PIPES, etc.), and phosphate buffered saline (PBS).

Generally, the standard substance contained in the calibrator is a substance having essentially the same or similar structure and properties as the test substance. The present inventors found that a complex comprising: an oligopeptide comprising an epitope of the polypeptide aggregate; a hydrophilic polymer chain not comprising the epitope; and a carrier particle (hereinafter also referred to as “the complex of the present embodiment”) can be used as a standard substance corresponding to the polypeptide aggregate. In the method for measuring of the present embodiment, a calibrator comprising the complex of the present embodiment is used.

The sample may contain a plurality of types of polypeptide aggregates. Which polypeptide aggregate to measure can be arbitrarily selected. In the method for measuring of the present embodiment, a calibrator comprising a complex corresponding to each of the polypeptide aggregates to be measured is used. That is, for one type of polypeptide aggregate, a calibrator comprising one type of corresponding complex is used.

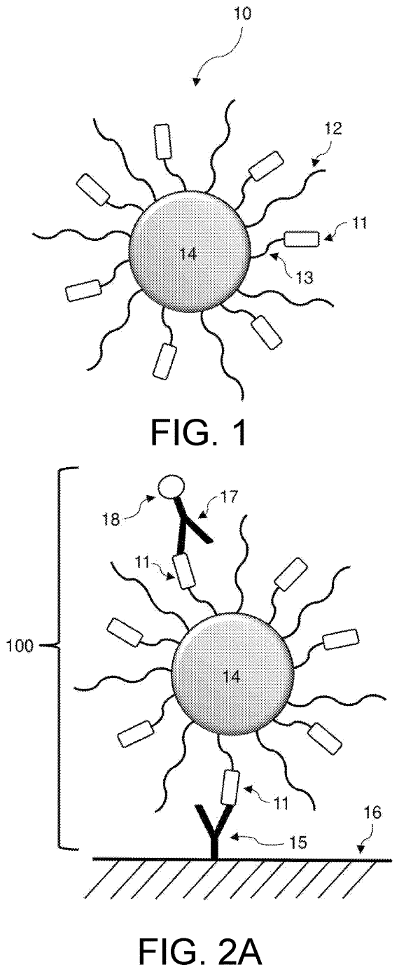

An example of the complex of the present embodiment is shown in FIG. 1, but the present invention is not limited to this example. In the figure, 10 denotes the complex. 11 denotes an oligopeptide comprising an epitope of the polypeptide aggregate (hereinafter also simply referred to as “oligopeptide”). 12 denotes a hydrophilic polymer chain not comprising the epitope of the polypeptide aggregate (hereinafter also simply referred to as “hydrophilic polymer chain”). 13 denotes a linker to be described later. 14 denotes a carrier particle. In the complex 10 in FIG. 1, a plurality of oligopeptides 11 are immobilized on a surface of the carrier particle 14 via the linker 13. The oligopeptide may be immobilized on a surface of the carrier particle without a linker. By immobilizing a plurality of oligopeptides on one carrier particle, the complex 10 can be a molecule mimicking the polypeptide aggregate. Further, a plurality of hydrophilic polymer chains 12 are immobilized on a surface of the carrier particle 14. This is expected to improve the dispersibility of the complex in solution. Furthermore, as shown in Example 3 described later, the presence of the hydrophilic polymer chain improves the storage stability of the complex of the present embodiment.

The oligopeptide comprising an epitope of the polypeptide aggregate is a molecule corresponding to a monomer polypeptide in the polypeptide aggregate which is the test substance. Preferably, a plurality of oligopeptide molecules are immobilized on the surface of the carrier particle contained in the complex of the present embodiment. The epitope of the polypeptide aggregate in the oligopeptide can be an epitope recognized by an antibody that specifically binds to the polypeptide aggregate. The number of epitopes in the oligopeptide can be one or more. The oligopeptide may be a molecule consisting of the epitope of the polypeptide aggregate.

The oligopeptide may be either a natural oligopeptide or a synthetic oligopeptide. The method for producing the oligopeptide itself is known, and examples include the Fmoc solid-phase synthesis method and the Boc solid-phase synthesis method. The length (number of amino acid residues) of the oligopeptide is, for example, 5 residues or more, 6 residues or more, 7 residues or more, 8 residues or more, 9 residues or more, or 10 residues or more. The length (number of amino acid residues) of the oligopeptide is, for example, 50 residues or less, 45 residues or less, 42 residues or less, 40 residues or less, 30 residues or less, 20 residues or less, or 15 residues or less.

The structure and amino acid sequence of the epitope of the polypeptide aggregate are not particularly limited. The amino acid sequence of the epitope may be, for example, the full-length sequence or a partial sequence of the monomer polypeptide of the polypeptide aggregate. Preferably, the amino acid sequence of the epitope is a partial sequence of the monomer polypeptide. The partial sequence of the monomer polypeptide is an amino acid sequence of a region consisting of a plurality of consecutive amino acid residues in the full-length sequence of the monomer polypeptide. That is, the epitope of the polypeptide aggregate can be a fragment of the monomer polypeptide. The amino acid sequence of the monomer polypeptide can be obtained from known databases such as GenBank, PDB, EMBL, and DDBJ.

For example, when the polypeptide aggregate is an amyloid β aggregate, the oligopeptide preferably comprises a partial sequence of the amyloid β peptide. The amyloid β peptide is a polypeptide produced by the cleavage of amyloid β precursor protein (APP) by β-secretase and γ-secretase. The amyloid β peptide is typically a polypeptide consisting of 39 to 43 amino acids. The term “amyloid β peptide” as used herein includes amyloid β peptide known in the art. Preferably, the amyloid β peptide is Aβ40 (DAEFRHDSGYEVHHQKLVFFAEDVGSNKGAIIGLMVGGVV: SEQ ID NO: 1) or Aβ42 (DAEFRHDSGYEVHHQKLVFFAEDVGSNKGAIIGLMVGGVVIA: SEQ ID NO: 2). The partial sequence of the AB peptide can be, for example, the same amino acid sequence as the epitope recognized by a monoclonal antibody that specifically binds to the Aβ peptide (hereinafter also referred to as “anti-AB monoclonal antibody”). The anti-Aβ monoclonal antibody itself is known and commercially available.

Examples of epitopes recognized by commercially available anti-AB monoclonal antibodies include:
A region of the Aβ peptide from position 1 to 16 (epitope recognized by the clone 82E1 antibody),
A region of the Aβ peptide from position 3 to 8 (epitope recognized by the clone 6E10 antibody),
A region of the AB peptide from position 4 to 10 (epitope recognized by the clone WO-2 antibody),
A region of the Aβ peptide from position 1 to 8 (epitope recognized by the clone 2H4 antibody),
A region of the AB peptide from position 36 to 42 (epitope recognized by the clone H31L21 antibody),
A region of the AB peptide from position 33 to 42 (epitope recognized by the clone G2-11 antibody),
A region of the AB peptide from position 33 to 42 (epitope recognized by the clone 16C11 antibody),
A region of the AB peptide from position 34 to 42 (epitope recognized by the clone 21F12 antibody),
A region of the AB peptide from position 35 to 40 (epitope recognized by the clone 1A10 antibody).

A preferred oligopeptide comprises a region comprising an amino acid sequence of the N-terminal side of the amyloid β peptide as the epitope. The amino acid sequence of the N-terminal side of the amyloid β peptide is, for example, the amino acid sequence of a region consisting of at least 5 consecutive residues in the amino acid sequence of position 1 to 20 of SEQ ID NO: 1 or the amino acid sequence of position 1 to 21 of SEQ ID NO: 2. Examples of such an epitope include the region from position 1 to 16, the region from position 3 to 8, the region from position 4 to 10, and the region from position 1 to 8 of the amyloid β peptide.

The oligopeptide is preferably immobilized on a surface of the carrier particle by a covalent bond. In this case, the N-terminal amino acid residue of the oligopeptide and the carrier particle may be joined by a covalent bond. Alternatively, the C-terminal amino acid residue of the oligopeptide and the carrier particle may be joined by a covalent bond. The term “covalent bond” as used herein also includes an M-S bond formed by the reaction of a metal (M) and a sulfhydryl group (hereinafter referred to as “SH group”). Examples of the metal forming the M-S bond include gold, platinum, silver, copper, and nickel. The mode of binding between the oligopeptide and the carrier particle will be described later.

In order to immobilize the oligopeptide on the carrier particle, the oligopeptide may further comprise a molecule having a predetermined functional group. Preferably, a molecule having a predetermined functional group is attached to the N-terminal side or C-terminal side of the oligopeptide. Examples of the molecule having a predetermined functional group include cysteine, a linker, and a combination thereof. Cysteine has an SH group as the predetermined functional group. By attaching cysteine to the N-terminal side or C-terminal side of the oligopeptide by an amide bond, the oligopeptide can have an SH group at its N-terminal side or C-terminal side.

In the complex of the present embodiment, it is preferable to include a linker between the oligopeptide and the carrier particle. A preferred linker is a bifunctional linker. The bifunctional linker itself is known and is commercially available as a cross-linking reagent. The bifunctional linker is a molecule consisting of a spacer arm and two functional groups. The bifunctional linker has a functional group at each end of the spacer arm. The spacer arm consists of a linear molecule such as, for example, a hydrocarbon or polyethylene glycol. A preferred spacer arm is a polyethylene glycol chain (hereinafter also referred to as “PEG chain”). The length of the spacer arm is not particularly limited. For example, the length of the spacer arm may be 10 Å or more, 12 Å or more, 15 Å or more, 17 Å or more, or 17.6 Å or more. Also, the length of the spacer arm may be 120 Å or less, 110 Å or less, 105 Å or less, 100 Å or less, or 96 Å or less. The spacer arm can be, for example, a PEG chain represented by the following formula (I).

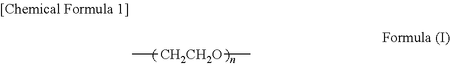

(wherein n is an integer from 1 to 24)

When attaching the bifunctional linker to the oligopeptide, it is preferable to use a heterobifunctional linker having a functional group for binding to the N-terminal amino acid residue or C-terminal amino acid residue of the oligopeptide (hereinafter also referred to as “functional group 1”) and a functional group for binding to the surface of the carrier particle (hereinafter also referred to as “functional group 2”). In the heterobifunctional linker, functional group 1 and functional group 2 are different from each other. Examples of functional group 1 include an amino group, a carboxy group, an N-hydroxysuccinimide (NHS) ester group, and a maleimide group. Functional group 2 can be appropriately determined depending on the material of the carrier particle and the functional groups present on the surface. Examples of functional group 2 include an SH group, an amino group, a carboxy group, and an NHS ester group.

The hydrophilic polymer chain not comprising the epitope of the polypeptide aggregate is present on the carrier particle together with the oligopeptide comprising an epitope of the polypeptide aggregate. The complex of the present embodiment has at least one hydrophilic polymer chain on the carrier particle. As described above, the presence of the hydrophilic polymer chain improves the storage stability of the complex of the present embodiment. Since the hydrophilic polymer chain does not comprise the epitope of the polypeptide aggregate, it does not bind to an antibody that specifically binds to the polypeptide aggregate.

The hydrophilic polymer chain is composed of a hydrophilic polymer having a straight chain or a branch. Preferably, the hydrophilic polymer chain is composed of a straight chain hydrophilic polymer. Examples of the hydrophilic polymer include polyethylene glycol, polypropylene glycol, polyamide, polyacrylamide, polyvinyl alcohol, and polyacrylic acid. Preferably, the hydrophilic polymer chain is a PEG chain. The hydrophilic polymer chain itself is known and commercially available.

In order to immobilize the hydrophilic polymer chain on the carrier particle, it is preferable that any one end of the hydrophilic polymer chain has a predetermined functional group. The predetermined functional group is the same as the aforementioned functional group 2. Examples of the predetermined functional group include an SH group, an amino group, a carboxy group, and an NHS ester group. In the hydrophilic polymer chain, the end without the predetermined functional group can be, for example, hydrogen, a hydroxy group, or an unsubstituted alkyl group having 1 to 6 carbon atoms with a straight chain or a branch. A hydrophilic polymer chain having one functional group is commercially available as, for example, a monofunctional PEG reagent.

The molecular weight of the hydrophilic polymer chain is, for example, about 500 or more, about 550 or more, about 600 or more, about 700 or more, about 800 or more, about 900 or more, about 1000 or more, about 1100 or more, about 1200 or more, about 1300 or more, about 1400 or more or about 1500 or more. The molecular weight of the hydrophilic polymer chain is, for example, about 50000 or less, about 45000 or less, about 40000 or less, about 35000 or less, about 30000 or less, about 21000 or less, about 20000 or less, about 18000 or less, about 15000 or less, about 12000 or less, about 11000 or less, about 10000 or less, about 9000 or less, about 8000 or less, about 7000 or less, about 6000 or less, about 5000 or less, about 4000 or less, about 3900 or less, about 3800 or less, about 3700 or less, about 3600 or less or about 3500 or less. In another embodiment, the molecular weight of the hydrophilic polymer chain is, for example, 500 or more, 550 or more, 600 or more, 700 or more, 800 or more, 900 or more, 1000 or more, 1100 or more, 1200 or more, 1300 or more, 1400 or more or 1500 or more. The molecular weight of the hydrophilic polymer chain is, for example, 50000 or less, 45000 or less, 40000 or less, 35000 or less, 30000 or less, 21000 or less, 20000 or less, 18000 or less, 15000 or less, 12000 or less, 11000 or less, 10000 or less, 9000 or less, 8000 or less, 7000 or less, 6000 or less, 5000 or less, 4000 or less, 3900 or less, 3800 or less, 3700 or less, 3600 or less or 3500 or less.

The molecular weight of the hydrophilic polymer chain can be, for example, the average molecular weight or the most abundant molecular weight. The average molecular weight of the hydrophilic polymer chain can be measured by gel permeation chromatography (GPC). The most abundant molecular weight of the hydrophilic polymer chain can be measured by mass spectrometry. Examples of mass spectrometry include infusion mass spectrometry, liquid chromatography-mass spectrometry (LC-MS), time-of-flight mass spectrometry (TOF-MS), and matrix-assisted laser desorption/ionization mass spectrometry (MALDI-MS). The conditions for mass spectrometry can be appropriately determined. The most abundant molecular weight is the most frequently contained molecular weight among the analysis results of the molecular weight of the hydrophilic polymer chain measured by mass spectrometry. When a commercially available hydrophilic polymer chain is used, the molecular weight may be the value disclosed by the manufacturer or supplier.

The “carrier particle” is a solid phase for holding the oligopeptide comprising an epitope of the polypeptide aggregate and the hydrophilic polymer chain not comprising the epitope. That is, in the complex of the present embodiment, the oligopeptide comprising an epitope of the polypeptide aggregate and the hydrophilic polymer chain are immobilized on a surface of the carrier particle.

The carrier particle may be any insoluble granular substance capable of immobilizing the oligopeptide and the hydrophilic polymer chain. The material of the carrier particle is not particularly limited. For example, it can be selected from metal, organic polymer compound, inorganic compound, biopolymer, and the like. Examples of the metal include gold, platinum, silver, copper, and nickel. Examples of the organic polymer compound include latex, polystyrene, and polypropylene. Examples of the inorganic compound include magnetic material (iron oxide, chromium oxide, ferrite, etc.), silica, alumina, and glass. Examples of the biopolymer include insoluble agarose, insoluble dextran, gelatin, and cellulose. Two or more of these may be used in combination. For example, a carrier particle comprising a core which is a non-metallic particle and a metal layer covering the surface of the core may be used.

The shape of the carrier particle is not particularly limited. Examples of the shape include a sphere, an ellipsoid, a cuboid, a cube, a cylinder, a pyramid, or a shape close to any of these. The average particle diameter of the carrier particle is, for example, 5 nm or more, preferably 10 nm or more. The average particle diameter of the carrier particle is, for example, 20 nm or less, preferably 10 nm or less. As used herein, the term “particle diameter” means diameter. For example, the average particle diameter of the carrier particle is a volume-based median diameter measured by an apparatus capable of measuring the particle size distribution of nanometer-scale particles (hereinafter also referred to as “nanoparticles”). An example of such an apparatus is the nanoparticle size distribution analyzer “SALD-7500nano” (Shimadzu Corporation). When a commercially available carrier particle is used, the average particle diameter may be the value disclosed by the manufacturer or supplier. The preferred carrier particle is a gold nanoparticle.

The complex of the present embodiment can be produced by immobilizing the oligopeptide and the hydrophilic polymer chain on a surface of the carrier particle. In the complex of the present embodiment, it is preferable that each of the oligopeptide and the hydrophilic polymer chain is immobilized on the surface of the carrier particle by a covalent bond. Immobilization by a covalent bond can be performed by a known method of modifying a solid phase surface with a functional molecule. For example, when the entirety or the surface of the carrier particle is a metal such as gold, platinum, silver, copper, or nickel, the carrier particle can immobilize the oligopeptide and the hydrophilic polymer chain having an SH group. As shown in the reaction scheme below, the metal (M) carrier particle reacts with the SH group to form an M-S bond. In the reaction scheme below, R is an oligopeptide or a hydrophilic polymer chain.

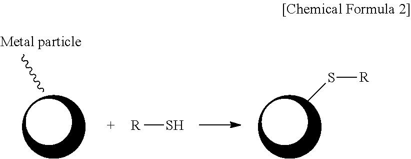

When a carboxy group is present on the surface of the carrier particle, the carrier particle can immobilize the oligopeptide and the hydrophilic polymer chain having an amino group. As shown in the reaction scheme below, first, the carboxy group of the carrier particle is activated using a carbodiimide such as 1-ethyl-3-(3-dimethylaminopropyl) carbodiimide (EDC). The carbodiimide group of EDC reacts with the carboxy group to form an o-acylisourea ester. The o-acylisourea ester is an unstable ester. Therefore, it readily reacts with the amino group of the oligopeptide or the hydrophilic polymer chain to form an amide bond. In the reaction scheme below, R is the oligopeptide or the hydrophilic polymer chain.

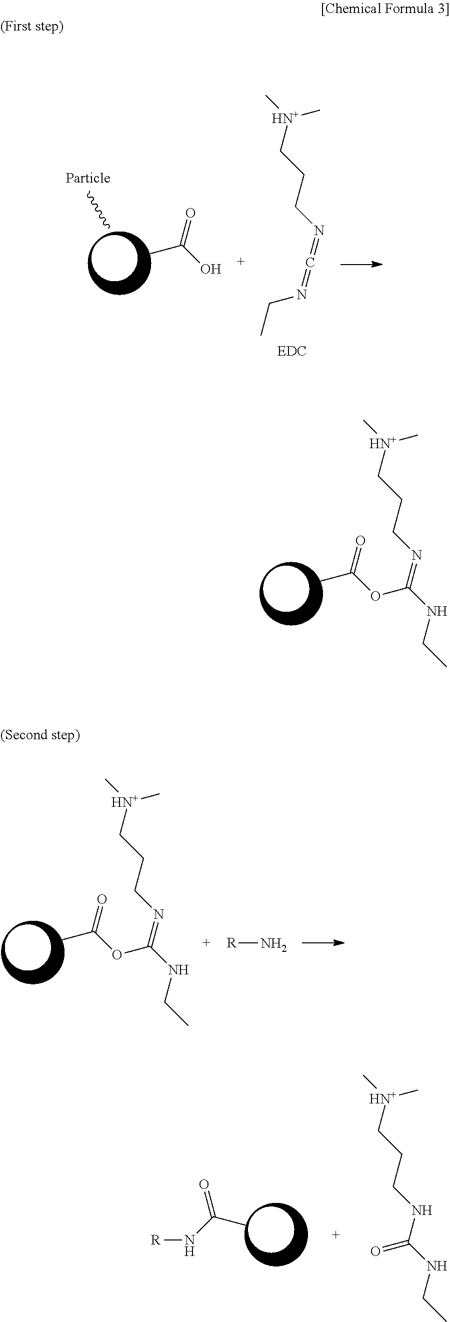

When an amino group is present on the surface of the carrier particle, the carrier particle can immobilize the oligopeptide and the hydrophilic polymer chain having an NHS ester group. As shown in the reaction scheme below, the NHS ester group of the oligopeptide or the hydrophilic polymer chain reacts with the amino group of the carrier particle to form an amide bond. In the reaction scheme below, R is the oligopeptide or the hydrophilic polymer chain.

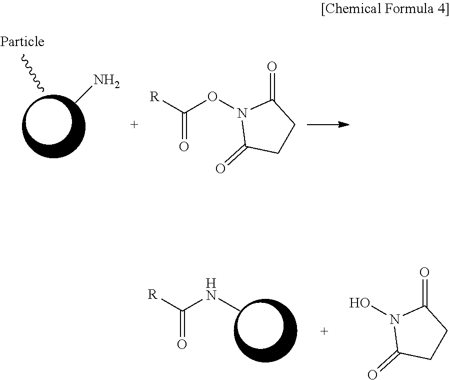

For the measurement of the complex in the calibrator and the polypeptide aggregate in the sample, a substance that can specifically bind to the polypeptide aggregate is used. Examples of such substances include antibodies and aptamers. Preferably, it is an antibody. As used herein, the term “antibody” includes not only the immunoglobulin form but also antibody fragments. Examples of antibody fragments include Fab, F(ab′)₂, F(ab′), Fv, Fd, domain antibody (dAb), single-chain antibody (scFv), reduced IgG (rIgG), diabody, and VHH. The antibody may be either a monoclonal antibody or a polyclonal antibody. A commercially available antibody may be used as the antibody that specifically binds to the polypeptide aggregate. Measurement using an antibody is called an immunoassay.

In the measurement method of the present embodiment, it is preferable to measure the complex in the calibrator and the polypeptide aggregate in the sample, respectively, by immunoassay. The type of immunoassay is not particularly limited. The immunoassay can be selected from known methods such as enzyme-linked immunosorbent assay (ELISA), immunoprecipitation, and immune complex transfer method (see Japanese Patent Application Laid-Open No. 1-254868). The type of ELISA is not particularly limited, and may be any of the sandwich method, competitive method, direct method, indirect method, and the like. The sandwich method is preferred. The immunoassay may be performed using a commercially available automated immunoassay analyzer such as the HISCL (registered mark) series (Sysmex Corporation) or HI-1000 (Sysmex Corporation). The measurement result by immunoassay is preferably an optical index that is visible or measurable by a machine. Examples of the optical index include luminescence intensity, fluorescence intensity, absorbance, turbidity, and color intensity.

The concentration of the complex of the present embodiment in the calibrator is known. Here, the concentration of the complex of the present embodiment refers to the protein concentration (e.g., in units of pg/mL). Therefore, information regarding the concentration of the polypeptide aggregate can be obtained based on the measurement result of the complex in the calibrator and the measurement result of the polypeptide aggregate in the sample. The information regarding the concentration of the polypeptide aggregate can be presented qualitatively, quantitatively, or semi-quantitatively. Qualitative information is information indicating the presence or absence of the polypeptide aggregate. Quantitative information is numerical information such as a numerical value obtained by the measuring instrument (hereinafter also referred to as “raw data”) or a value calculated from the numerical value. Examples of the value calculated from the raw data include a value obtained by subtracting the value of the negative control sample or the background value from the raw data. The value of the concentration of the polypeptide aggregate can be determined based on the quantitative information. Semi-quantitative information is information that shows the concentration of the polypeptide aggregate stepwise using phrases, numbers (indicating classes), colors, or the like. For example, phrases such as “below the limit of detection”, “low”, “medium”, or “high” may be used.

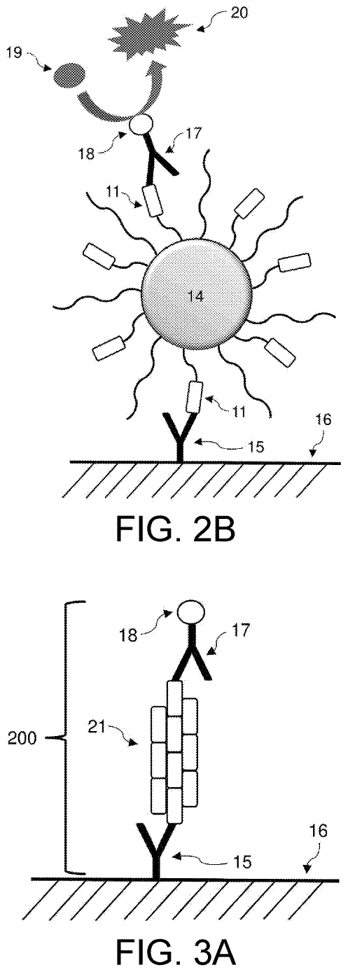

Referring to FIG. 2A and FIG. 2B, an example of measuring the complex in the calibrator by sandwich ELISA using a capture antibody and a detection antibody that specifically bind to the polypeptide aggregate will be described. In this example, the complex in the calibrator is the complex 10 shown in FIG. 1. However, the present invention is not limited to this example. Referring to FIG. 2A, in the measurement of the complex in the calibrator, first, a first immune complex 100 comprising the complex 10, the capture antibody 15, the detection antibody 17, and the labeling substance 18 is formed on a solid phase 16. The capture antibody, the detection antibody, the labeling substance, and the solid phase will be described later. In FIG. 2A, the labeling substance 18 is pre-bound to the detection antibody 17. The labeling substance 18 is an enzyme.

The first immune complex 100 is formed by mixing the calibrator comprising the complex 10, the capture antibody 15, and the detection antibody 17 comprising the labeling substance 18. The step of forming the first immune complex is preferably performed in a solution comprising the complex of the present embodiment, the capture antibody, and the detection antibody. The capture antibody 15 binds to the epitope in the oligopeptide 11 present on the surface of the carrier particle 14. Similarly, the detection antibody 17 binds to the epitope in the oligopeptide 11 present on the surface of the carrier particle 14. The complex has a plurality of oligopeptides. Therefore, as shown in FIG. 2A, the capture antibody and the detection antibody can each bind to a different oligopeptide. Next, the first immune complex 100 is formed on the solid phase 16 by contacting the solution comprising the first immune complex 100 with the solid phase 16 on which the capture antibody 15 can be immobilized. Alternatively, a solid phase on which the capture antibody is pre-immobilized may be used. In this case, the first immune complex is formed on the solid phase by contacting the calibrator comprising the complex, the solid phase with the immobilized capture antibody, and the detection antibody comprising the labeling substance.

Referring to FIG. 2B, the labeling substance 18 in the first immune complex 100 formed on the solid phase 16 is contacted with the substrate 19 of the labeling substance 18. By this contact, the labeling substance 18 in the first immune complex 100 and the substrate 19 react to generate a signal 20. The signal emitted from the first immune complex is referred to as the “first signal”. As shown in FIG. 2B, the signal 20 is generated from the detection antibody 17 and the labeling substance 18 bound to the oligopeptide 11 contained in the complex 10. Therefore, the complex can be measured by detecting the first signal.

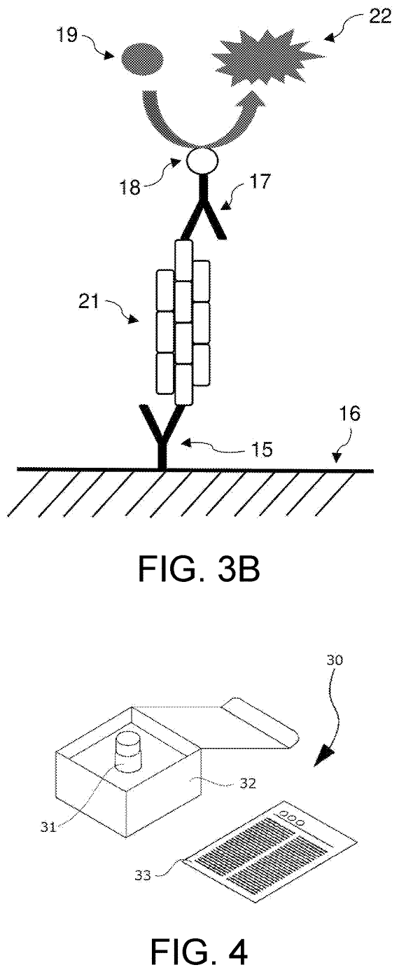

Referring to FIG. 3A and FIG. 3B, an example of measuring the polypeptide aggregate in the sample by sandwich ELISA using a capture antibody and a detection antibody that specifically bind to the polypeptide aggregate will be described. However, the present invention is not limited to this example. Referring to FIG. 3A, in the measurement of the polypeptide aggregate in the sample, first, a second immune complex 200 comprising the polypeptide aggregate 21, the capture antibody 15, the detection antibody 17, and the labeling substance 18 is formed on a solid phase 16. In FIG. 3A, the labeling substance 18 is pre-bound to the detection antibody 17. The labeling substance 18 is an enzyme.

The second immune complex 200 is formed by mixing the sample that may comprise the polypeptide aggregate 21, the capture antibody 15, and the detection antibody 17 comprising the labeling substance 18. The step of forming the second immune complex is preferably performed in a solution comprising the polypeptide aggregate, the capture antibody, and the detection antibody. The capture antibody 15 binds to the epitope in the polypeptide aggregate 21. Similarly, the detection antibody 17 binds to the epitope in the polypeptide aggregate 21. The polypeptide aggregate 21 is composed of a plurality of monomer polypeptides. Therefore, as shown in FIG. 3A, the capture antibody and the detection antibody can each bind to a different epitope. Next, the second immune complex 200 is formed on the solid phase 16 by contacting the solution comprising the second immune complex 200 with the solid phase 16 on which the capture antibody 15 can be immobilized. Alternatively, a solid phase on which the capture antibody is pre-immobilized may be used. In this case, the second immune complex is formed on the solid phase by contacting the sample that may comprise the polypeptide aggregate 21, the solid phase with the immobilized capture antibody, and the detection antibody comprising the labeling substance.

Referring to FIG. 3B, the labeling substance 18 in the second immune complex 200 formed on the solid phase 16 is contacted with the substrate 19 of the labeling substance 18. By this contact, the labeling substance 18 in the second immune complex 200 and the substrate 19 react to generate a signal 22. The signal emitted from the second immune complex is referred to as the “second signal”. As shown in FIG. 3B, the signal 22 is generated from the detection antibody 17 and the labeling substance 18 bound to the polypeptide aggregate 21. Therefore, the polypeptide aggregate can be measured by detecting the second signal.

As used herein, “detecting a signal” includes qualitatively detecting the presence or absence of the signal, quantifying the intensity of the signal, and semi-quantitatively detecting the intensity of the signal. Semi-quantitative detection means showing the intensity of the signal stepwise, such as “no signal generated”, “weak”, “medium”, or “strong”. The value obtained by quantifying the intensity of the signal is also referred to as the “signal measurement value”. The detection result of the first signal can be used as the measurement result of the complex in the calibrator. The detection result of the second signal can be used as the measurement result of the polypeptide aggregate in the sample. For example, when quantifying the intensity of the first signal, the first signal measurement value itself or a value acquired from the measurement value can be used as the measurement value of the complex. Also, when quantifying the intensity of the second signal, the second signal measurement value itself or a value acquired from the measurement value can be used as the measurement value of the polypeptide aggregate. Examples of the value acquired from the signal measurement value include a value obtained by subtracting the measurement value of the negative control sample or the background value from the signal measurement value.

As described above, the concentration (protein concentration) of the complex of the present embodiment contained in the calibrator is known. Therefore, the relationship between the measurement value of the first signal and the protein concentration is clear. From this, the concentration of the polypeptide aggregate contained in the sample can be obtained from the measurement value of each of the first signal and the second signal. Preferably, a plurality of calibrators having different concentrations of the complex of the present embodiment are measured to obtain the measurement value of the first signal of each calibrator. By obtaining the measurement value of the first signal from the plurality of calibrators, a calibration curve can be created. The calibration curve can be created by, for example, plotting the measurement values of the first signal obtained from the plurality of calibrators on an XY plane with the concentration of the complex (protein concentration) on the X-axis and the signal measurement value on the Y-axis, and obtaining a straight line or a curve by a known method such as the method of least squares. By fitting the measurement value of the second signal to this calibration curve, the concentration of the polypeptide aggregate in the sample can be obtained. Furthermore, a regression equation representing the obtained calibration curve may be acquired. The obtained regression equation allows the measurement value of the second signal to be converted into the concentration of the polypeptide aggregate in the sample.

In the above example, the sample is measured after the calibrator is measured, but the order of measurement is not particularly limited. The calibrator may be measured after the sample is measured. Alternatively, the sample and the calibrator may be measured in parallel.

In the measurement method of the present embodiment, B/F (Bound/Free) separation to remove unreacted free components that have not formed a complex may be performed between the formation of the first immune complex and the measurement of the first signal. Also, B/F separation to remove unreacted free components that have not formed a complex may be performed between the formation of the second immune complex and the measurement of the second signal. Unreacted free components refer to components that do not constitute the immune complex. Examples include the capture antibody and the detection antibody that did not bind to the complex of the present embodiment or the polypeptide aggregate. The means of B/F separation is not particularly limited, but if the solid phase is a particle, B/F separation can be done by recovering only the solid phase that captured the immune complex by centrifugation. If the solid phase is a container such as a microplate or a microtube, B/F separation can be done by removing the liquid comprising the unreacted free components. In the case where the solid phase is a magnetic particle, B/F separation can be done by aspirating and removing the liquid comprising the unreacted free components with a nozzle while the magnetic particles are magnetically constrained by a magnet, which is preferable from the viewpoint of automation. After removing the unreacted free components, the solid phase that captured the immune complex may be washed with an appropriate aqueous medium such as PBS.

The capture antibody and the detection antibody are antibodies that specifically bind to the polypeptide aggregate by recognizing the epitope of the polypeptide aggregate in the sample. Since the complex of the present embodiment has an oligopeptide comprising the epitope of the polypeptide aggregate, the capture antibody and the detection antibody can also bind to the complex of the present embodiment. The capture antibody is an antibody for immobilizing the polypeptide aggregate and the complex of the present embodiment on a solid phase. The detection antibody is an antibody for providing a detectable signal via a labeling substance. It is preferable that the detection antibody is not immobilized on the solid phase. The epitopes recognized by the capture antibody and the detection antibody may be the same. Alternatively, the epitopes recognized by the capture antibody and the detection antibody may be different from each other.

The capture antibody and the detection antibody can be appropriately determined depending on the type of the polypeptide aggregate to be detected and the epitope in the oligopeptide of the complex of the present embodiment. For example, when the polypeptide aggregate is an Aβ aggregate, the capture antibody and the detection antibody can be determined from the above anti-Aβ monoclonal antibodies depending on the epitope in the oligopeptide of the complex of the present embodiment.

The solid phase may be any insoluble carrier capable of immobilizing the capture antibody. The material of the solid phase is not particularly limited. For example, it can be selected from the above-mentioned organic polymer compounds, inorganic compounds, biopolymers, and the like. The solid phase may be used in combination of two or more of these. The shape of the solid phase is not particularly limited, and examples include particles, microplates, microtubes, test tubes, and membranes. Among these, particles (especially magnetic particles) and microplates are preferred.

The mode of immobilization of the capture antibody on the solid phase is not particularly limited. For example, the capture antibody and the solid phase may be directly bound. Alternatively, the capture antibody and the solid phase may be indirectly bound via another substance. Examples of direct binding include adsorption by hydrophobic interaction and covalent bonding using functional groups. Examples of indirect binding include binding via a combination of biotins and avidins. The capture antibody and the solid phase can be indirectly bound via the binding of the biotins and the avidins by pre-modifying the capture antibody with biotins and pre-binding avidins to the solid phase.

As used herein, “biotins” include biotin and its analogs. Examples of analogs of biotin include desthiobiotin and biocytin. As used herein, “avidins” include avidin and its analogs. Examples of analogs of avidin include streptavidin, avidin-like protein derived from Tamogitake mushroom (Tamavidin (registered mark)), bradavidin, and rizavidin.

The labeling substance is not particularly limited, as long as a detectable signal is generated. The labeling substance may be, for example, a substance that itself generates a signal (hereinafter also referred to as a “signal generating substance”). The labeling substance may also be a substance that catalyzes the reaction of other substances to generate a signal. Examples of the signal generating substance include a fluorescent substance and a radioisotope. Examples of the substance that catalyzes the reaction of other substances to generate a detectable signal include an enzyme. Examples of enzymes include alkaline phosphatase, peroxidase, β-galactosidase, and luciferase. Examples of fluorescent substances include fluorescent dyes such as fluorescein isothiocyanate (FITC), rhodamine, and Alexa Fluor (registered mark), and fluorescent proteins such as GFP. Examples of radioisotopes include ¹²⁵I, ¹⁴C, and ³²P. Enzymes are preferred as the labeling substance, and alkaline phosphatase and peroxidase are particularly preferred.

The method for detecting the signal itself is known in the art. A measurement method corresponding to the type of signal derived from the above labeling substance can be appropriately selected. For example, when the labeling substance is an enzyme, a signal such as light or color generated by reacting with the substrate for the enzyme is measured. Known instruments such as a spectrophotometer or a microplate reader can be used for measuring the signal.

The substrate for the enzyme can be appropriately selected from known substrates depending on the type of the enzyme. When the enzyme is alkaline phosphatase, for example, the following substances are used as the substrate:
CDP-Star (registered mark) (4-chloro-3-(methoxyspiro[1, 2-dioxetane-3, 2′-(5′-chloro) tricyclo[3.3.1.13,7]decan]-4-yl) phenyl phosphate disodium)
CSPD (registered mark) (3-(4-methoxyspiro[1, 2-dioxetane-3, 2′-(5′-chloro) tricyclo[3.3.1.13,7]decan]-4-yl) phenyl phosphate disodium)
5-bromo-4-chloro-3-indolyl phosphate (BCIP)
5-bromo-6-chloro-indolyl phosphate disodium
p-nitrophenyl phosphate

When the enzyme is peroxidase, for example, the following substances are used as the substrate:
Luminol and its derivatives
2, 2′-azinobis (3-ethylbenzothiazoline-6-sulfonic acid ammonium) (ABTS)
1, 2-phenylenediamine (OPD)
3, 3′,5, 5′-tetramethylbenzidine (TMB)

When the labeling substance is a radioisotope, the radiation as a signal can be measured using known instruments such as a scintillation counter. When the labeling substance is a fluorescent substance, the fluorescence as a signal can be measured using known instruments such as a microplate reader. The excitation wavelength and fluorescence wavelength can be appropriately determined depending on the type of the fluorescent substance.

The detection antibody preferably comprises a labeling substance. The detection antibody and the labeling substance may be directly bound. Alternatively, the detection antibody and the labeling substance may be indirectly bound via another substance. Examples of direct binding between the detection antibody and the labeling substance include a covalent bond. Binding of the detection antibody and the labeling substance by a covalent bond can be performed using a commercially available cross-linker or labeling kit. A detection antibody to which a labeling substance is bound by a covalent bond is also referred to as a labeled antibody. Examples of indirect binding between the detection antibody and the labeling substance include binding between the detection antibody and a labeled antibody (labeled secondary antibody) that specifically binds to the detection antibody.

A further embodiment provides a calibrator (hereinafter also referred to as “calibrator of the present embodiment”) for obtaining information regarding the concentration of a polypeptide aggregate in a sample. The calibrator of the present embodiment is a reagent comprising a complex of an oligopeptide comprising an epitope of the polypeptide aggregate, a hydrophilic polymer chain not comprising the epitope, and a carrier particle. The details of the complex contained in the calibrator of the present embodiment are the same as those described for the complex of the present embodiment.

The complex contained in the calibrator of the present embodiment may be a solid (e.g., powder, lyophilized product, etc.). Alternatively, the complex contained in the calibrator of the present embodiment may be liquid (e.g., suspension, emulsion, etc.). When the complex is a solid, the complex is dispersed in the above aqueous medium and used. Preferred aqueous media are PBS or Good's buffer.

The calibrator of the present embodiment may comprise additives. Examples of additives include a protein stabilizer, an inhibitor for heterophile antibodies, a preservative, and inorganic salts. Examples of the protein stabilizer include bovine serum albumin (BSA) and sodium caseinate. Examples of the inhibitor for heterophile antibodies include mouse IgG and goat IgG. Examples of preservatives include sodium azide and thimerosal. Examples of inorganic salts include sodium chloride and potassium chloride.

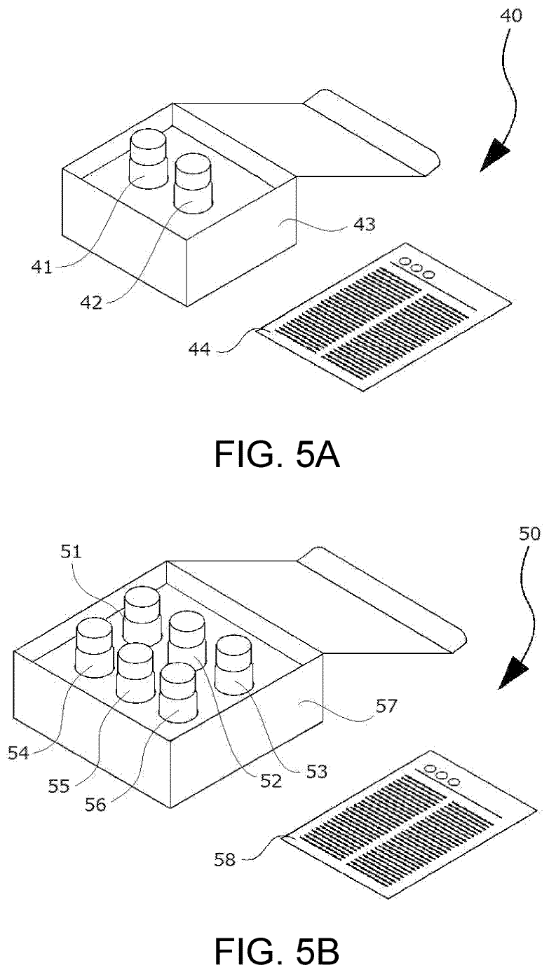

A calibrator in one embodiment consists of a single reagent at the time of provision to the user. The calibrator consisting of a single reagent can be diluted by the user with an aqueous solvent to prepare a plurality of reagents. The plurality of reagents comprise the complex of the present embodiment at mutually different concentrations. An example of a calibrator consisting of a single reagent is shown in FIG. 4. In FIG. 4, 30 denotes a kit comprising the calibrator. 31 denotes a first container housing the calibrator comprising the complex of the present embodiment. 32 denotes a packaging box. 33 denotes a package insert. The package insert describes, for example, the method of use, storage method, and composition of the calibrator of the present embodiment.

A calibrator in another embodiment is a reagent set consisting of a plurality of reagents. The plurality of reagents comprise the complex of the present embodiment at mutually different concentrations. The number of reagents contained in the calibrator is not particularly limited. The number of reagents can be selected from, for example, 2, 3, 4, 5, 6, and 7. The concentration of the complex in each reagent contained in the calibrator is not particularly limited, but it is preferably set so that the above calibration curve can be created. In the calibrator of the present embodiment, the reagent with the highest complex concentration may comprise the complex of the present embodiment at a concentration that is 2 times or more and 1000 times or less the concentration of the reagent with the lowest concentration. The calibrator of the present embodiment may comprise a control in which the complex concentration is 0. Such a control can be, for example, the above aqueous solvent.

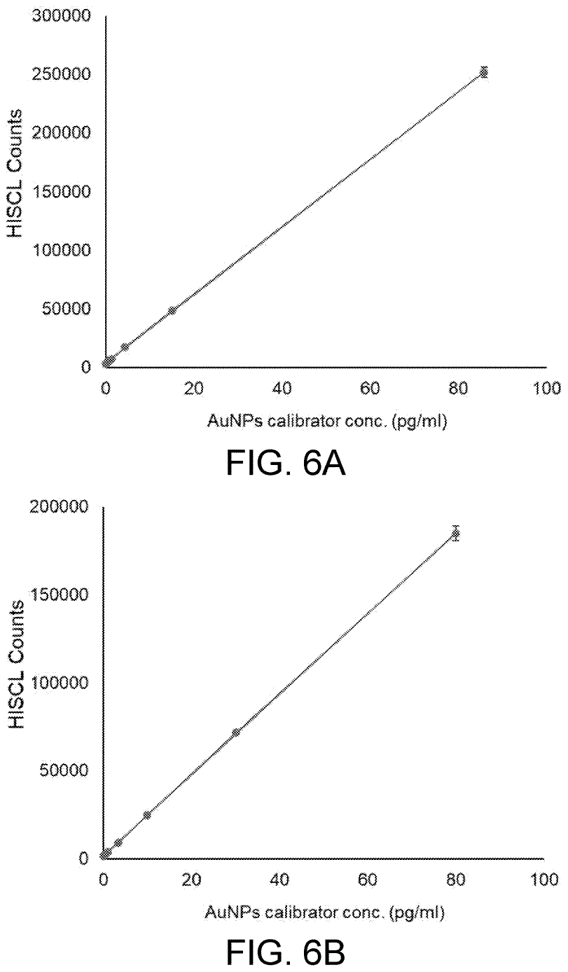

An example of the calibrator of the present embodiment which is a reagent set is shown in FIG. 5A. In FIG. 5A, 40 denotes a kit comprising the calibrator. 41 denotes a first container housing a first calibrator comprising the complex of the present embodiment. 42 denotes a second container housing a second calibrator comprising the complex of the present embodiment at a different concentration from the first calibrator. 43 denotes a packaging box. 44 denotes a package insert.

Another example of the calibrator of the present embodiment which is a reagent set is shown in FIG. 5B. In FIG. 5B, 50 denotes a kit comprising the calibrator. 51 denotes a first container housing a first calibrator with the highest complex concentration. 52 denotes a second container housing a second calibrator with the second highest complex concentration. 53 denotes a third container housing a third calibrator comprising the complex of the present embodiment with the third highest complex concentration. 54 denotes a fourth container housing a fourth calibrator with the fourth highest complex concentration. 55 denotes a fifth container housing a fifth calibrator with the lowest complex concentration. 56 denotes a sixth container housing an aqueous solvent not comprising the complex of the present embodiment. 57 denotes a packaging box. 58 denotes a package insert.

A kit comprising the calibrator which is a reagent set may further comprise a container housing the capture antibody, a container housing the solid phase, a container housing the detection antibody comprising an enzyme as the labeling substance, and a container housing the substrate for the enzyme.

A further embodiment provides the use of the complex of the oligopeptide comprising the epitope of the polypeptide aggregate, the hydrophilic polymer chain not comprising the epitope, and the carrier particle for the production of a calibrator for obtaining information regarding the concentration of the polypeptide aggregate in a sample.

A further embodiment provides the use of the oligopeptide comprising the epitope of the polypeptide aggregate, the hydrophilic polymer chain not comprising the epitope, and the carrier particle for the production of a complex used for obtaining information regarding the concentration of the polypeptide aggregate in a sample.

Hereinafter, the present invention will be described in detail by examples, but the present invention is not limited to these examples.

### Example 1: Preparation of Calibrators

### (1) Preparation of PICUP Calibrator

Aβ42 oligomer was prepared by PICUP as follows. First, Aβ42 monomer was dissolved in 10 mM sodium hydroxide to obtain an Aβ42 peptide solution with a concentration of 0.5 mg/mL. This peptide solution was sonicated for 5 minutes with a bath sonicator, and then centrifuged at 16,000 g for 10 minutes at 4° C. Ru(Bpy), a cross-linking agent, and ammonium persulfate (APS) were added to the 150 M Aβ42 peptide solution in a ratio of 1:2:5 (Aβ42:Ru(Bpy):APS) to prepare the PICUP reaction solution. The reaction solution was irradiated with a laser at an output of 15 mW for 12.5 seconds to perform the cross-linking reaction. 1 M L-cysteine was immediately added to the reaction solution to a final concentration of 50 mM to stop the reaction. The reaction solution was concentrated to 350 μL using Amicon 10K (10,000 g for 10 minutes at 4° C.) and purified at 4° C. using a Superdex 200 10/300 GL column. The fraction of Aβ42 oligomer was collected and concentrated using Amicon 10K (10,000 g for 10 minutes at 4° C.). The obtained solution was stored as the Aβ42 oligomer. The oligomer concentration in the solution was measured by absorbance using a spectrophotometer NanoDrop (Thermo Scientific). The Aβ42 oligomer solution was serially diluted with MES buffer (pH 7.0, 1% BSA, 0.1 M NaCl) to prepare five solutions. The oligomer concentrations of these solutions were measured with NanoDrop. The concentrations were 0.65, 1.18, 4.36, 14.94, and 85.77 pg/mL, respectively. These solutions were used as PICUP calibrators for the subsequent experiments.

### (2) Preparation of Gold Nanoparticle Calibrator

### (2.1) Preparation of Oligopeptide Comprising A3 Aggregate Epitope

A synthetic peptide consisting of the amino acid sequence of positions 1 to 8 of Aβ42 (DAEFRHDS: SEQ ID NO: 3) (hereinafter also referred to as “Aβ₁₋₈”) was used as the epitope of the Aβ aggregate. NH₂-PEG6-COOH was used as the PEG linker. This was a heterobifunctional PEG reagent having an amino group and a carboxy group at each end of the PEG chain (—(CH₂ O)₆—). Aβ₁₋₈-PEG6-COOH was obtained by reacting the carboxy group of the serine residue of Aβ₁₋₈ with the amino group of NH₂-PEG6-COOH. Aβ₁₋₈-PEG6-Cys was obtained by reacting the carboxy group of Aβ₁₋₈-PEG6-COOH with the amino group of cysteine. The preparation of Aβ₁₋₈-PEG6-Cys was commissioned to GenScript Inc.

### (2.2) Preparation of the Complex

Aβ₁₋₈-PEG6-Cys was dissolved in PBS to obtain a peptide solution of 2 mg/mL. “mPEG-SH, 550”, “mPEG-SH, 3.4k”, “mPEG-SH, 10k”, or “mPEG-SH, 20k” (Biopharma PEG Scientific Inc.) was used as the hydrophilic polymer chain not comprising the epitope. mPEG-SH was a monofunctional PEG reagent having a methyl group and an SH group at each end of the PEG chain. Various mPEG-SH were measured by a mass spectrometer MALDI-8030 (Shimadzu Corporation). As a result, the most abundant molecular weights of the various mPEG-SH were as follows.
mPEG-SH, 550:554.76
mPEG-SH, 3.4k:3682.72
mPEG-SH, 10k:10235.91
mPEG-SH, 20k:20911.01

Various mPEG-SH were dissolved in PBS to obtain a solution of 4 mg/mL. The peptide solution (20 μL), the mPEG-SH solution (10 μL), and PBS (10 μL) were added to a 1.5 mL tube and mixed. A suspension of gold nanoparticles (diameter 13 to 17 nm, Sigma-Aldrich Inc.) (160 μL) was added thereto and mixed with a vortex mixer. The mixture was reacted for 5 minutes at room temperature. After adding 20% BSA/PBS (20 μL) to the tube, it was centrifuged at 12, 000 g for 5 minutes. The supernatant was removed, and 5% BSA/PBS (200 μL) was added. Thereby, a complex in which Aβ₁₋₈-PEG6-Cys and the PEG chain were immobilized on the surface of the gold nanoparticle was obtained.

### (2.3) Determination of Complex Concentration by Immunoassay Using PICUP Calibrator

The above complex solution was diluted 10000 times with 5% BSA/PBS and used as a sample. The immunoassay was performed using the fully automated immunoassay analyzer HISCL (registered mark)-5000 (Sysmex Corporation). Biotin-modified 82E1 F(ab′) was used as the capture antibody. ALP-modified 82E1 F(ab′) was used as the detection antibody. Here, 82E1 F(ab′) is the F(ab′) of the antibody of clone 82E1 (Immuno-Biological Laboratories Co., Ltd.). The composition of each reagent used in the immunoassay is shown below.
R1 Reagent (Reagent comprising capture antibody): pH 7.0 solution comprising 50 fmol/assay biotin-modified 82E1 F(ab′), 0.1 M MES, 0.1 M NaCl, 1% BSA, 0.1% NaN₃
R2 Reagent (Reagent comprising magnetic particles): pH 7.0 solution comprising 0.5% streptavidin-bound magnetic particles
R3 Reagent (Reagent comprising detection antibody): pH 7.5 solution comprising 100 fmol/assay ALP-modified 82E1 F(ab′), 0.1 M Tris-HCl, 0.15 M NaCl, 0.1% NaN₃

HISCL R4 Reagent (Sysmex Corporation) was used as the R4 Reagent (measurement buffer). HISCL R5 Reagent (Sysmex Corporation) was used as the R5 Reagent (ALP substrate solution).

The measurement procedure using HISCL-5000 was as follows. After adding R1 reagent (50 μL) to a cuvette, each reagent of the PICUP calibrator (30 μL) or the sample (30 μL) was added and mixed. R2 reagent (30 μL) was added to the cuvette and mixed. The magnetic particles in the mixture were collected magnetically and the supernatant was removed. HISCL washing solution (300 μL) was added to wash the magnetic particles, and then R3 reagent (100 μL) was added. The supernatant was removed, and R4 reagent (50 μL) and R5 reagent (100 μL) were added to the cuvette and mixed. Then, the luminescence intensity (count) was obtained as the signal measurement value. The measurement was performed 3 times, and the average value of the 3 measurements was obtained. A calibration curve was created from the average values of the measurements of each reagent of the PICUP calibrator. The concentration of the sample was obtained by fitting the sample measurement value to this calibration curve. The average value of the sample concentration was 100 pg/mL.

### (2.4) Measurement of Gold Nanoparticle Calibrator

The complex solution whose concentration was determined to be 100 pg/mL was serially diluted by the gravimetric method to concentrations of 80, 30, 10, 3.3, 1.0, and 0.5 pg/mL. MES buffer was used for dilution. Specifically, a portion of the 100 pg/mL complex solution was taken, its weight was measured with a precision balance, and it was diluted based on the weight to achieve the above concentrations. Each diluted solution is referred to as C6, C5, C4, C3, C2, and C1. Furthermore, MES buffer was used as C0. C0 to C6 were frozen with liquid nitrogen and stored at −80° C. until use. Table 1 shows the concentration of the complex contained in each of C0 to C6.

TABLE 1

| Reagents | Concentration (pg/mL) |
|---|---|
| C0 | 0.0 |
| C1 | 0.5 |
| C2 | 1.0 |
| C3 | 3.3 |
| C4 | 10.0 |
| C5 | 30.0 |
| C6 | 80.2 |

The complexes were measured using HISCL-5000 in the same manner as described above, using the aforementioned C0 to C6 as samples. The measurement was performed three times, and the average value of the three measurements was obtained. For each sample, a calibration curve was created from the concentration determined by the gravimetric method and the average measured value. From each calibration curve, it was found that the complex measurements obtained by immunoassay correlated extremely well with the complex concentrations determined by the gravimetric method. The above C0 to C6 were used as gold nanoparticle calibrators for the subsequent experiments.

### Example 2: Evaluation of the Calibration Curve of the Gold Nanoparticle Calibrator

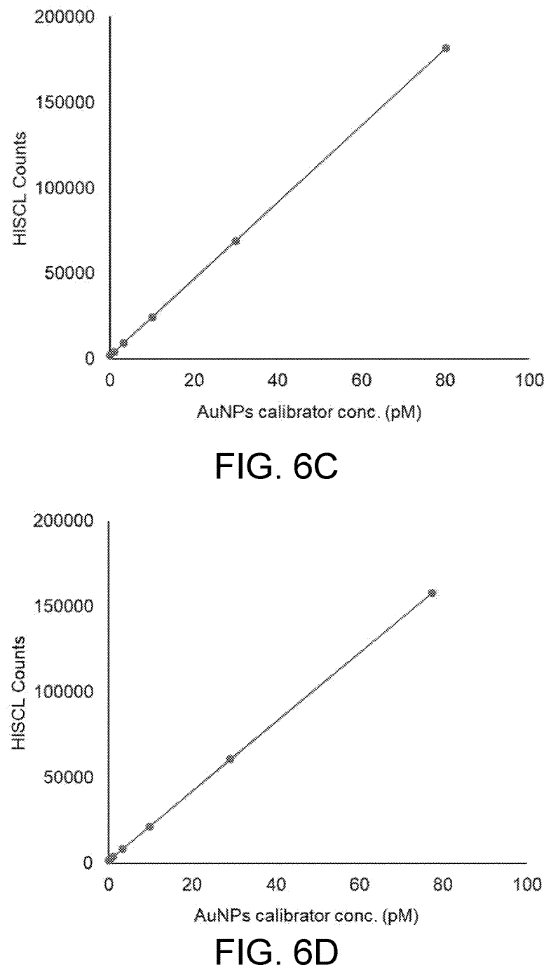

C6 prepared in Example 1 was thawed by standing under running water at room temperature for 5 minutes. The thawed C6 was serially diluted with MES buffer to prepare samples with different concentrations. The weight of each diluted sample at the time of preparation was measured with a precision balance, and the dilution factor was determined from the weight value. Each sample was measured with HISCL-5000 in the same manner as in Example 1. For comparison, a reagent containing a complex that did not have a hydrophilic polymer chain without an epitope (hereinafter referred to as the “Comparative Example Complex”) was prepared and measured under the same conditions. The Comparative Example Complex was obtained by immobilizing only Aβ₁₋₈-PEG6-Cys on the surface of the gold nanoparticles, in the same manner as in Example 1, except that PBS was used instead of the mPEG-SH solution. The measurement was performed three times, and the average value of the three measurements was obtained. A calibration curve was created for the average value and the diluted concentration of each sample using logistic regression. The validity of the calibration curve was evaluated based on a criterion that the coefficient of determination R² during fitting was 0.99 or higher. The calibration curve created based on the measurement of the reagent containing the Comparative Example Complex is shown in FIG. 6A. The calibration curves created based on the measurement of the calibrators (PEG chain molecular weights: 550, 3.4k, 10k, and 20k) prepared in Example 1 are shown in FIG. 6B to FIG. 6E, respectively. Furthermore, the regression equations and R² values for each calibration curve shown in FIG. 6A to FIG. 6E are shown below.

### FIG. 6A:

y=2891.9x+3943.4(R²=0.9999)

### FIG. 6B:

y=2292.3x+1852.7(R²=1)

### FIG. 6C:

y=2241.3x+1954(R²=1)

### FIG. 6D:

y=2022.2x+1620.7(R²=1)

### FIG. 6E:

y=1944.1x+1556.7(R²=1)

As can be seen from FIG. 6A, even when using the Comparative Example Complex, the complex measurements obtained by immunoassay correlated well with the complex concentrations determined by the gravimetric method. As can be seen from FIG. 6B to FIG. 6E, regardless of the molecular weight of the non-epitope-containing PEG chain, the complex measurements obtained by immunoassay correlated extremely well with the complex concentrations determined by the gravimetric method. This measurement range was capable of quantifying Aβ42 in plasma.

Example 3: Evaluation of the Storage Stability of the Gold Nanoparticle Calibrator C0 and C5 prepared in Example 1 were thawed by standing under running water at room temperature for 5 minutes. The thawed C0 and C5 were incubated in an incubator at 4° C. or 37° C. for 0 or 72 hours. Subsequently, each sample was measured with HISCL-5000 in the same manner as in Example 1. The measurement was performed three times, and the average value of the three measurements was obtained. To account for the stability of the calibrators, the thawing time of the calibrators was adjusted so that all samples were measured on the same day. A reagent containing the Comparative Example Complex was prepared, and its storage stability was evaluated under the same conditions.

The storage stability was evaluated based on the residual activity calculated from the average measured values of C0 and C5 after standing at a temperature of 4° C. or 37° C. The residual activity was obtained as follows. For C0 and C5 after standing at 4° C. for 72 hours, the average measured value of C0 was subtracted from the average measured value of C5 (the resulting value is referred to as “C5(4° C.)”). For C0 and C5 after standing at 37° C. for 72 hours, the average measured value of C0 was subtracted from the average measured value of C5 (the resulting value is referred to as “C5(37° C.)”). The residual activity (%) was calculated by substituting each value into the following equation.

$$Residual Activity ( \% ) = [ C 5 ( 37 ° C . ) / C 5 ( 4 ° C . ) ] \times 100$$

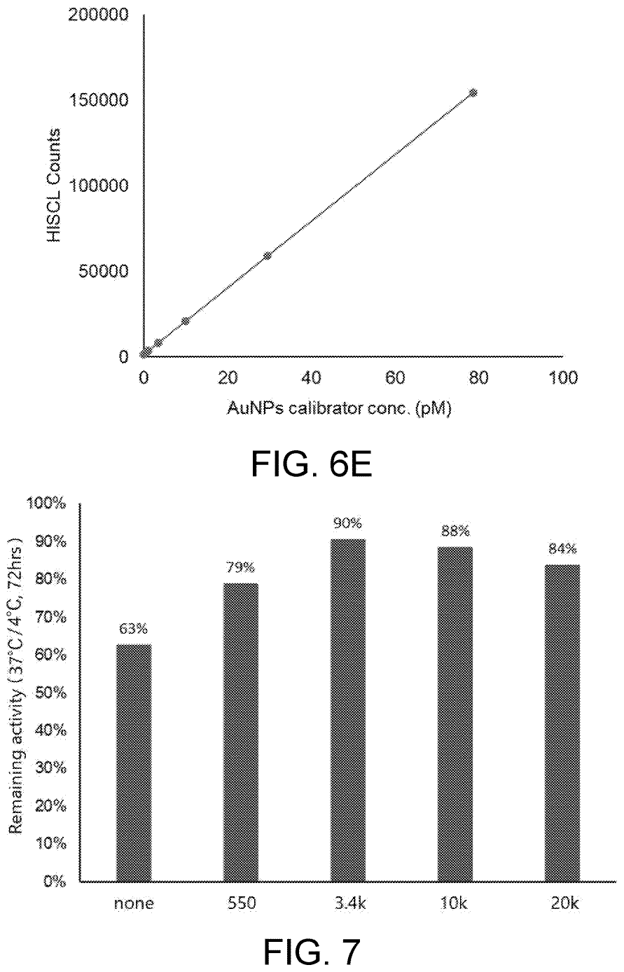

The results are shown in FIG. 7. In the figure, “none” represents the Comparative Example Complex. “550”, “3.4k”, “10k”, and “20k” indicate the molecular weights of the PEG chains contained in the complex within C5. As can be seen from FIG. 7, C5 of the calibrator prepared in Example 1 showed higher residual activity than the reagent containing the Comparative Example Complex. This result suggests that the storage stability of the calibrator can be improved by using a complex in which not only the oligopeptide containing the epitope of the polypeptide aggregate but also a hydrophilic polymer chain without an epitope are immobilized on the carrier particle.

What is claimed is:

1. A method for measuring a polypeptide aggregate in a sample, comprising obtaining information regarding a concentration of the polypeptide aggregate in the sample using a calibrator,
wherein the calibrator comprises a complex comprising: an oligopeptide comprising an epitope of the polypeptide aggregate; a hydrophilic polymer chain not comprising the epitope; and a carrier particle.

2. The method according to claim 1, wherein
the calibrator is a reagent set comprising a plurality of reagents each comprising the complex, and
the concentration of the complex differs among the plurality of reagents.

3. The method according to claim 1, wherein in the complex, the oligopeptide comprising the epitope of the polypeptide aggregate and the hydrophilic polymer chain not comprising the epitope are immobilized on a surface of the carrier particle.

4. The method according to claim 1, wherein
the complex in the calibrator and the polypeptide aggregate in the sample are each measured by immunoassay, and
information regarding the concentration of the polypeptide aggregate is obtained based on the measurement results of the complex and the polypeptide aggregate in the sample.

5. The method according to claim 4, comprising:
obtaining a measurement value of the complex using the calibrator, a capture antibody specifically binding to the epitope, a detection antibody specifically binding to the epitope, and a labeling substance;
obtaining a measurement value of the polypeptide aggregate in the sample using the sample, the capture antibody, the detection antibody, and the labeling substance; and
obtaining information regarding the concentration of the polypeptide aggregate in the sample based on the measurement value of the complex and the measurement value of the polypeptide aggregate.

6. The method according to claim 5, wherein a calibration curve is created from the measurement value of the complex, and
the concentration of the polypeptide aggregate in the sample is obtained from the calibration curve and the measurement value of the polypeptide aggregate.

7. The method according to claim 1, wherein the sample is a blood sample or cerebrospinal fluid.

8. The method according to claim 1, wherein the polypeptide aggregate is an amyloid β aggregate.

9. The method according to claim 8, wherein the oligopeptide comprises a region comprising an amino acid sequence of the N-terminal side of the amyloid β peptide as the epitope.

10. The method according to claim 1, wherein the hydrophilic polymer chain is a polyethylene glycol chain.

11. The method according to claim 10, wherein a molecular weight of the polyethylene glycol chain is from about 500 to about 21,000.

12. The method according to claim 1, wherein the carrier particle is a gold nanoparticle.

13. A calibrator for obtaining information regarding a concentration of a polypeptide aggregate in a sample, comprising a complex comprising: an oligopeptide comprising an epitope of the polypeptide aggregate; a hydrophilic polymer chain not comprising the epitope; and a carrier particle.

14. The calibrator according to claim 13, wherein the calibrator is a reagent set comprising a plurality of reagents each comprising the complex, and a concentration of the complex differs among the plurality of reagents.

15. A complex for use in the method according to claim 1, comprising: the oligopeptide comprising the epitope of the polypeptide aggregate; the hydrophilic polymer chain not comprising the epitope; and the carrier particle.

16. A method for producing a complex for use to obtain information regarding a concentration of a polypeptide aggregate in a sample, comprising immobilizing an oligopeptide comprising an epitope of the polypeptide aggregate and a hydrophilic polymer chain not comprising the epitope on a surface of a carrier particle.

17. The method according to claim 16, wherein in the complex, the oligopeptide comprising the epitope of the polypeptide aggregate and the hydrophilic polymer chain not comprising the epitope are immobilized on a surface of the carrier particle.

18. The method according to claim 16, wherein the polypeptide aggregate is an amyloid β aggregate and the oligopeptide comprises a region comprising an amino acid sequence of the N-terminal side of the amyloid β peptide as the epitope.

19. The method according to claim 16, wherein the hydrophilic polymer chain is a polyethylene glycol chain.

20. The method according to claim 19, wherein a molecular weight of the polyethylene glycol chain is from about 500 to about 21,000.
